(19) United States
(12) Patent Application Publication
(10) Pub. No.: US 2005/0113690 A1
(43) Pub. Date: May 26, 2005

(54) Methods and systems for providing portable device extended resources

(76) Inventors: Nahi Halmann, Milwaukee, WI (US); Anne Lindsay Hall, New Berlin, WI (US); Shinichi Amemiya, Tokyo (JP)

Correspondence Address: Dean D. Small;Armstrong Teasdale LLP, Suite 2600, One Metropolitan Square, St. Louis, MO, 63102, US

(21) Appl. No.: 10/891,210

(22) Filed: Jul. 14, 2004

Related U.S. Application Data
(60) Provisional application No. 60/524,941, filed on Nov. 25, 2003.

(51) Int. Cl. A61B 8/00; A61B 8/14

(52) U.S. Cl. 600/437; 600/444

(57) ABSTRACT

A method of ultrasound imaging is provided. The method includes coupling a first ultrasound probe to a portable ultrasound-imaging device, the probe configured to provide a first predetermined set of functions, scanning a volume of interest using the portable ultrasound-imaging device at a first transmit power level capability to acquire ultrasound image data, coupling the portable ultrasound-imaging device to a resource extension device, processing at least a portion of the ultrasound image data using a ultrasound-imaging device processor, and processing at least a portion of the ultrasound image data using a resource extension device processor.

### CROSS-REFERENCE TO RELATED APPLICATIONS

This application claims priority to and the benefit of the filing date of U.S. Provisional Application No. 60/524,941 filed on Nov. 25, 2003 and which is hereby incorporated by reference in its entirety.

### BACKGROUND OF THE INVENTION

This invention relates generally to ultrasound systems and, more particularly, to methods and systems for providing extended resources for portable ultrasound systems.

Ultrasound systems, and more particularly, medical ultrasound systems, are used for many different types of medical scanning procedures. These medical ultrasound systems allow for imaging organs and soft tissue structures in the human body. Ultrasound imaging is often preferred over other medical imaging modalities because it is real time, non-invasive, portable, and relatively low cost.

However, many known imaging techniques may require processing resources that exceed the processing resources available in portable imaging systems. Such systems may be capable of imaging patients in a relatively lower power mode and may only be capable of providing a display of a portion of the collected image data or only a preprocessed version of the collected image data. Moreover, the imaging system may be limited and provide reduced capability due to electrical power and processing power constraints on the available imaging system hardware.

### BRIEF DESCRIPTION OF THE INVENTION

In one aspect, a method of ultrasound imaging is provided. The method includes coupling a first ultrasound probe to a portable ultrasound-imaging device, the probe configured to provide a first predetermined set of functions, scanning a volume of interest using the portable ultrasound-imaging device at a first transmit power level capability to acquire ultrasound image data, coupling the portable ultrasound-imaging device to a resource extension device, processing at least a portion of the ultrasound image data using a ultrasound-imaging device processor, and processing at least a portion of the ultrasound image data using a resource extension device processor.

In another aspect, a portable ultrasound-imaging system is provided. The system includes an ultrasound-imaging device including an input port that is configured to receive at least one of a plurality of probes, each probe having at least one function that is different from a function of each other of the plurality of probes, a resource extension device removably couplable to the ultrasound-imaging device for at least one of adding an ultrasound-imaging capability to the ultrasound-imaging device and modifying an ultrasound-imaging capability of the ultrasound-imaging device, and a display for outputting ultrasound images.

### BRIEF DESCRIPTION OF THE DRAWINGS

FIG. 1 is a block diagram of an ultrasound-imaging device in accordance with one exemplary embodiment of the present invention;

FIG. 2 is block diagram of the exemplary ultrasound-imaging device shown in FIG. 1;

FIG. 3 is block diagram of the exemplary ultrasound-imaging device shown in FIG. 1, including a resource extension device.

FIG. 4 is a flow chart of an exemplary method of ultrasound-imaging that may be used with the ultrasound-imaging system shown in FIG. 3.

### DETAILED DESCRIPTION OF THE INVENTION

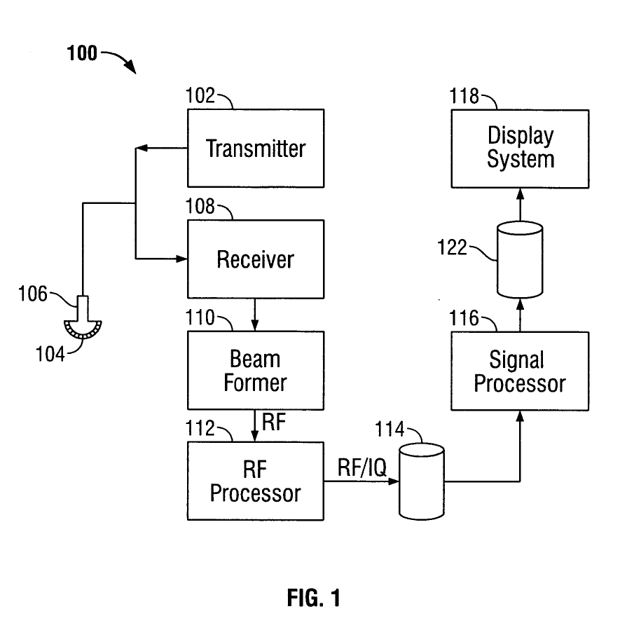

FIG. 1 is a block diagram of an exemplary ultrasound-imaging device 100. Ultrasound-imaging device 100 includes a transmitter 102 that drives a plurality of transducers 104 within a probe 106 to emit pulsed ultrasound signals into a body. A variety of geometries may be used. The ultrasound signals are back-scattered from density interfaces and/or structures in the body, like blood cells or muscular tissue, to produce echoes which return to transducers 104. A receiver 108 receives the echoes. The received echoes are passed through a beamformer 110, which performs beamforming and outputs a RF signal. The RF signal then passes through a RF processor 112. Alternatively, RF processor 112 may include a complex demodulator (not shown) that demodulates the RF signal to form IQ data pairs representative of the echo signals. The RF or IQ signal data may then be routed directly to a RF/IQ buffer 114 for temporary storage.

Ultrasound-imaging device 100 also includes a processor 116 to process the acquired ultrasound information (i.e., RF signal data or IQ data pairs) and prepare frames of ultrasound information for display on a display 118. Processor 116 is adapted to perform one or more processing operations according to a plurality of selectable ultrasound modalities on the acquired ultrasound information. In the exemplary embodiment, acquired ultrasound information is processed in real-time during a scanning session as the echo signals are received. In an alternative embodiment, the ultrasound information may be stored temporarily in RF/IQ buffer 114 during a scanning session and processed in less than real-time in a live or off-line operation.

Ultrasound-imaging device 100 may continuously acquire ultrasound information at a frame rate that exceeds fifty frames per second, which is approximately the perception rate of the human eye. The acquired ultrasound information is displayed on display 118 at a slower frame-rate. An image buffer 122 is included for storing processed frames of acquired ultrasound information that are not scheduled to be displayed immediately. In the exemplary embodiment, image buffer 122 is of sufficient capacity to store at least several seconds worth of frames of ultrasound information. The frames of ultrasound information are stored in a manner to facilitate retrieval thereof according to its order or time of acquisition. Image buffer 122 may include at least one memory device, such as, but not limited to, a read only memory (ROM), a flash memory, and/or a random access memory (RAM) or other known data storage medium.

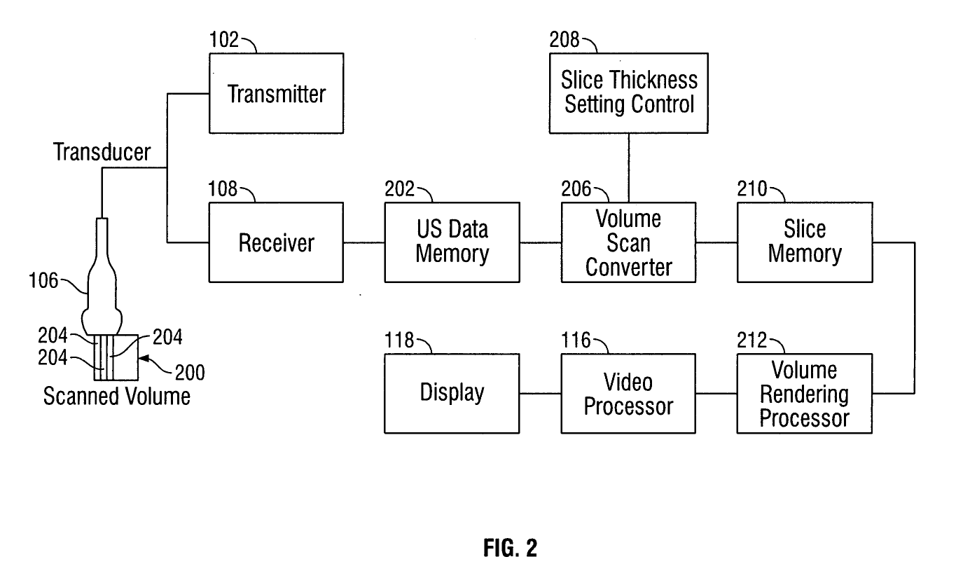

FIG. 2 is another block diagram of the exemplary ultrasound-imaging device 100 (shown in FIG. 1) that may be used to acquire and process ultrasound images. Ultrasound-imaging device 100 includes probe 106 connected to transmitter 102 and a receiver 108. Probe 106 transmits ultrasound pulses and receives echoes from structures inside of a scanned ultrasound volume 200. A memory 202 stores ultrasound data from receiver 108 derived from scanned ultrasound volume 200. Volume 200 may be obtained by various techniques, for example, but not limited to, 3D scanning, real-time 3D imaging, volume scanning, 2D scanning with transducers having positioning sensors, freehand scanning using a Voxel correlation technique, 2D scanning, and/or scanning with matrix array transducers.

Probe 106 is translated, such as, along a linear or arcuate path, while scanning a volume of interest. At each linear or arcuate position, probe 106 obtains a plurality of scan planes 204. Scan planes 204 are collected for a thickness, such as from a group or set of adjacent scan planes 204. Scan planes 204 are stored in memory 202, and then passed to a volume scan converter 206. In some embodiments, probe 106 may obtain lines instead of scan planes 204, and memory 202 may store lines obtained by probe 106 rather than scan planes 204. Volume scan converter 206 may receive scan lines obtained by probe 106 rather than scan planes 204. Volume scan converter 206 receives a slice thickness setting from a control input 208, which identifies the thickness of a slice to be created from scan planes 204. Volume scan converter 206 creates a data slice from multiple adjacent scan planes 204. The number of adjacent scan planes 204 that are obtained to form each data slice is dependent upon the thickness selected by slice thickness control input 208. The data slice is stored in a slice memory 210 and is accessed by a volume rendering processor 212. Volume rendering processor 212 performs volume rendering upon the data slice. The output of volume rendering processor 212 is transmitted to processor 116 and display 118.

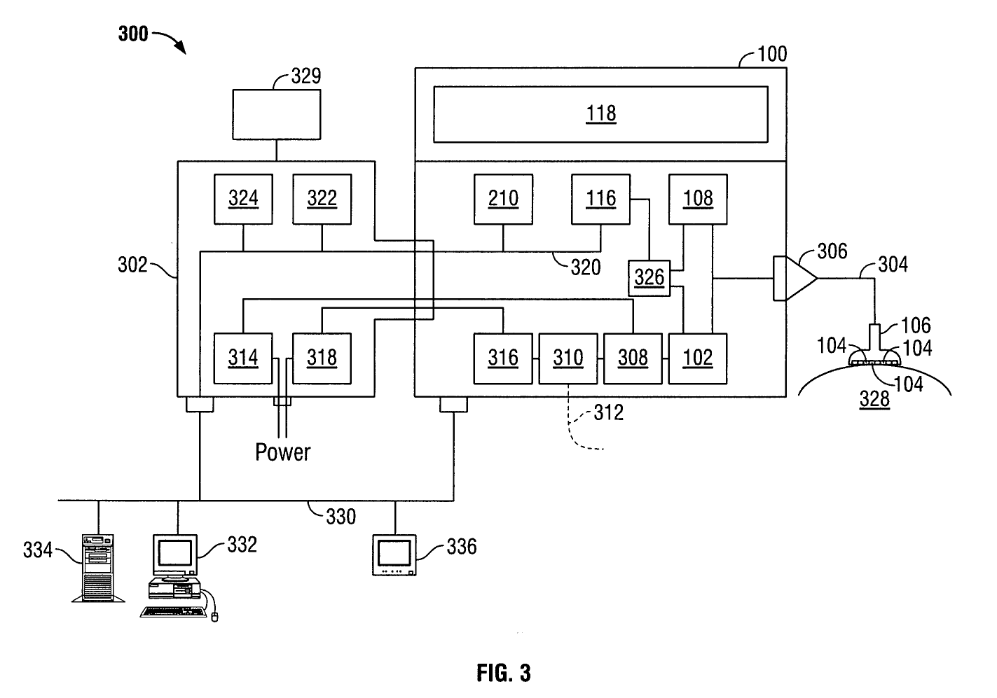

FIG. 3 is a block diagram 300 of the exemplary ultrasound-imaging device 100 (shown in FIG. 1) coupled to a resource extension device 302. Ultrasound-imaging device 100 may include a plurality of components that may be limited in their capability because the components are smaller to allow ultrasound-imaging device 100 to be portable. Resource extension device 302 in combination with ultrasound-imaging device 100 extends the capability of ultrasound-imaging device 100 when ultrasound-imaging device 100 and resource extension device 302 are coupled together. For example, ultrasound-imaging device 100 may only have a capability of a channel count of about thirty two, whereas when coupled to resource extension device 302, the channel count may be increased to about 512 or about 1024 channels, thereby extending the beamforming capabilities of ultrasound-imaging device 100. Ultrasound-imaging device 100 and resource extension device 302 may be mechanically coupled together, or may be communicatively coupled together while remaining physically separate. As used herein, portable means capable of mobile operation, such as, for example, of a size small enough to be hand-held while in operation. Ultrasound-imaging device 100 includes probe 106 that may be coupled to ultrasound-imaging device 100 through a cable 304 and a connector 306. Probe 106 may provide various functions that define a type of scan that optimizes the operational characteristics of probe 106. Therefore, a user may select among a variety of probes 106, wherein each different probe 106 may include at least one function or operational characteristic that is different from the functions or operational characteristics of other probes. For example, a particular probe 106 may include functions that make it advantageously suitable for fetal imaging, whereas a second probe 106 may include functions that make it advantageously suitable for cardiac imaging. Furthermore, a probe may include a motor for mechanical 3D image capture and resource extension device 302 may include a motor controller circuit for controlling the operate of 3D mechanical probes. Connector 306 is configured to couple a plurality of different types of probes 106 to ultrasound-imaging device 100. In addition, ultrasound-imaging device 100 may be configured to detect the type of probe 106 coupled to ultrasound-imaging device 100 and to display the type of probe on display 118. For example, ultrasound-imaging device 100 may detect a pin-to-pin electrical characteristic of connector 306 or probe 106 that is unique to each different type of probe 106 and/or ultrasound-imaging device 100 may detect a mechanical keying arrangement of connector 306 to facilitate determining the type of probe 106 coupled to connector 306.

Transmit signals are transmitted from transmitter 102 to transducers 104 through connector 306 and cable 304. Received echo signals are transmitted from transducers 104 to receiver 108 through cable 304 and connector 306. Transmitter 102 may receive transmit power from a power supply 308 located on-board ultrasound-imaging device 100. Power supply 308 may receive power of a first capability from an on-board power source 310. Source 310 may be, for example, a battery or other energy storage device, or source 310 may be powered by a user supplied source (not shown) through a portable cable 312. Power supply 308 may receive power of a second capability from a resource extension device power source 314, which provides an extended transmit power capability to transmitter 102 through power supply 308. Such extended power capability permits transducers 104 to transmit higher power ultrasound waves into the volume of interest, improving depth of penetration of the ultrasound waves into the volume of interest. A similar benefit may be achieved when supplying power from a user's supply through cable 312. When supplying power through cable 312, resource extension device 302 does not need to be coupled to ultrasound-imaging device 100 to achieve the higher power benefit as the user supplied source may replace power source 314 for supplying power supply 308. When coupled to resource extension device 302, a charging circuit 316 supplies charging current to source 310 that permits ultrasound-imaging device 100 to operate independent of resource extension device 302 for a predetermined period of time. In the exemplary embodiment, circuit 316 receives direct current (DC) power for charging from a resource extension device power supply 318. In an alternative embodiment, circuit 316 receives alternating current (AC) power for charging from resource extension device power supply 318.

Processor 116 may be coupled to memory 210 through a data path 320, such as, for example a data bus. Memory 210 may store an operating system (OS) that executes on processor 116 to control the operation of ultrasound-imaging device 100, and a variety of application programs for ultrasound imaging that operate under the control of the OS. Resource extension device 302 includes a processor 322 and an associated memory 324 that are coupled to data path 320, such that processor 116, memory 210, processor 322, and memory 324 may communicate. In the exemplary embodiment, data path 320 is a data channel that is USB (universal serial bus) standard compliant. In an alternative embodiment, data path 320 is a data channel that is IEEE 1394 standard compliant. Memory 324 may store a separate operating system (OS) from the OS stored in memory 210, that executes on processor 322 to control the operation of resource extension device 302, and a variety of application programs for ultrasound imaging that operate under the control of the operating system of processor 322. In addition, when ultrasound-imaging device 100 is coupled to resource extension device 302, the operating system of processor 322 may control the operation of ultrasound-imaging device 100 and resource extension device 302. As such, the operating system of processor 116 may only include a subset of the instructions and/or capabilities of the operating system of processor 322. Likewise, processor 116 may have a reduced processing capability relative to processor 322 and memory 210 may have a reduced storage capability relative to memory 324. The reduced capabilities of components of ultrasound-imaging device 100 relative to the capabilities of corresponding components of resource extension device 302 facilitates portability and ease of use of ultrasound-imaging device 100 but, easily permits expansion of the capabilities of ultrasound-imaging device 100 when ultrasound-imaging device 100 is coupled to resource extension device 302 by allowing components of one device to cooperate with components of the other device. Processor 116 is also coupled to receiver 108 and transmitter 102 through a scan control section 326.

In one embodiment, ultrasound-imaging device 100 includes only limited beamforming capabilities, for example, a channel count of at least one of transmit and receive is reduced relative the channel count when ultrasound-imaging device 100 is coupled to resource extension device 302. As such, ultrasound-imaging device 100 may continue to post-process the acquired data and resource extension device 302 may be utilized as a more sophisticated beamformer.

In operation in a portable mode, a probe 106 is selected from a plurality of available probes 106 and coupled to transmitter 102 and receiver 108 through connector 306. Probe 106 is used by abutting a face of probe 106 against an object to be imaged 328. Transmitter 102 and receiver 108 facilitate scanning the interior of object to be imaged 328 by a beam of pulsed ultrasound waves under the control of scan control section 326, and receive an echo of the ultrasound waves.

In one embodiment, ultrasound-imaging device 100 is used to acquire scan data, and additional processing, to perform, for example, but not limited to, volume/surface rendering, intra-media thickness measurements, and strain imaging is accomplished when ultrasound-imaging device 100 is coupled to resource extension device 302.

Scan control section 326 is controlled by processor 116 to perform various scans, such as, but not limited to, a B-mode imaging scan, a continuous wave scan, and a pulsed Doppler imaging scan, which may be displayed on display 118. The B-mode image represents a cross-sectional image of, for example, a tissue within object to be imaged 328. The pulsed Doppler image may represent a flow velocity distribution of, for example, blood flow within object to be imaged 328. In one embodiment, ultrasound-imaging device 100 is limited in capability such that only B-mode processing is preformed onboard ultrasound-imaging device 100 and resource extension device 302 is required for colorflow, Doppler, bflow, and codes processing. The image data captured by ultrasound-imaging device 100 may be viewed in real-time on display 118 or may be stored in memory 210, for example, in a first file format for later viewing or transfer to memory 324. First file format may be selected to facilitate compact storage of the image data in memory 210. Additionally, after coupling ultrasound-imaging device 100 to resource extension device 302, processor 322 may access the image data stored in memory 210 directly, or may process the image data from memory 324 after the image data has been transferred to memory 324. The image data may be stored in memory 324 in a second file format, for example that facilitates image data processing and viewing.

When ultrasound-imaging device 100 is operating independently from resource extension device 302, power supply 308 supplies power from source 310, which may be a rechargeable battery, to the components of ultrasound-imaging device 100. Accordingly, ultrasound-imaging device 100 can be used when it is uncoupled from resource extension device 302.

In operation, in an extended resource mode, such as, when ultrasound-imaging device 100 is coupled to resource extension device 302, processor 116 and processor 322 operate cooperatively to process image data according to predetermined instructions and input from a user. Processor 116 may operate to control both ultrasound-imaging device 100 and resource extension device 302 during a first time period when ultrasound-imaging device 100 and resource extension device 302 are coupled, and processor 322 may operate to control both ultrasound-imaging device 100 and resource extension device 302 during a second time period when ultrasound-imaging device 100 and resource extension device 302 are coupled, and during a third time period when ultrasound-imaging device 100 and resource extension device 302 are coupled each may control at least a portion of their respective devices.

The image data may be stored in an archive memory 329 to facilitate processing by caching data not immediately needed by ultrasound-imaging device 100 and resource extension device 302, for archiving, and/or for transfer to another system user (not shown). Archive memory 329 may be implemented as a hard disk drive (HDD) and/or a removable storage drive, such as, a floppy disk drive, a magnetic tape drive, or an optical disk drive. The image data file stored in archive memory 329 may be read as required or desired by the user, and displayed on the display 118.

Processor 116 and processor 322 may be configured to communicate with a data network 330 that may include, for example, a network terminal 332, and the image data may be uploaded to a server 334. Server 334 may also be used to download data and programs to ultrasound-imaging device 100 and/or resource extension device 302. For example, resource extension device 302 may detect when ultrasound-imaging device 100 is coupled to it and trigger an automatic synchronization of data between resource extension device 302 and/or ultrasound-imaging device 100, and a networked picture archiving communication system (PACS) system upon docking. Such synchronization may archive all the images that have been stored in the local hard-disk of ultrasound-imaging device 100, into the PACS system. Furthermore, ultrasound-imaging device 100 may automatically upload a list of patients that to be examined including each patient's demographic data. Ultrasound-imaging device 100 and resource extension device 302 may also display image data on an available monitor 336, such as, a television in a patient room. Accordingly, viewing images and/or real-time scan data on an available television screen may facilitate enhancing a diagnosis without sacrificing portability of ultrasound-imaging device 100.

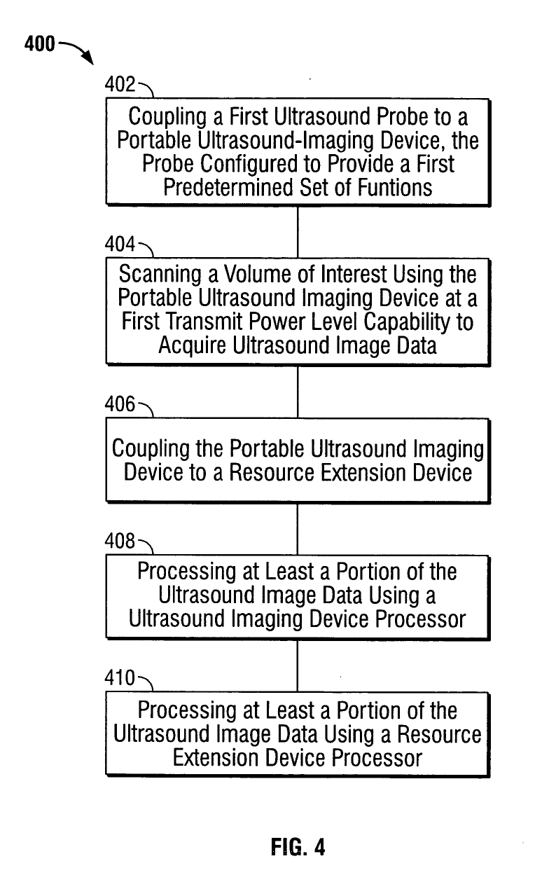

FIG. 4 is a flow chart of an exemplary method 400 of ultrasound imaging that may be used with system 300 (shown in FIG. 3). Method 400 includes coupling 402 a first ultrasound probe to a portable ultrasound-imaging device wherein the probe includes a first predetermined set of functions. Functions of the probe relate to characteristics, such as, but, not limited to, electrical, mechanical, and/or acoustic characteristics that make the probe more suitable for a particular type of scan. A volume of interest is scanned 404 using the portable ultrasound-imaging device at a first transmit power level capability to acquire ultrasound image data. The transmit power capability of the ultrasound-imaging device may depend on a user's power selection, a corresponding power level to a user selected type of scan, and whether the ultrasound-imaging device is coupled to the resource extension device. The resource extension device may increase the transmit power level capability of the ultrasound-imaging device and may include a motor controller circuit for driving a mechanical 3D probe. The portable ultrasound-imaging device may be coupled 406 to the resource extension device, thus making the extended resources of the resource extension device available for processing and displaying the acquired image data. System 300 (shown in FIG. 3) may then process 408 at least a portion of the acquired ultrasound image data using a ultrasound-imaging device processor, and process 410 at least a portion of the ultrasound image data using a resource extension device processor. In the exemplary embodiment, the processors of the ultrasound-imaging device and the resource extension device operate cooperatively to process the acquired image data, transfer the acquired image data from memory to a storage device or a network, and/or to display the acquired image data. In an alternative embodiment, the ultrasound-imaging device processor operates to process the acquired image data, transfer the acquired image data from memory to a storage device or a network, and/or to display the acquired image data. In another alternative embodiment, the resource extension device processor operates to process the acquired image data, transfer the acquired image data from memory to a storage device or a network, and/or to display the acquired image data.

Exemplary embodiments of systems and methods that facilitate extending the capabilities of a portable ultrasound-imaging device are described above in detail. A technical effect of the portable ultrasound systems and methods described herein include at least one of facilitating improving the portability of an ultrasound-imaging device while extending the image data collection, data processing, and image display capabilities of the ultrasound-imaging device.

The above-described methods and systems provide a cost-effective and reliable means for providing extended resources for portable ultrasound systems. More specifically, the methods and systems facilitate operating the ultrasound system in a portable mode and providing greater power and processing capability when coupled to a resource extension device. As a result, the methods and systems described herein facilitate monitoring patients in a variety of situational environments while maintaining portability and enhanced operational features in a cost-effective and reliable manner.

Exemplary embodiments of portable ultrasound systems are described above in detail. The systems are not limited to the specific embodiments described herein, but rather, components of each system may be utilized independently and separately from other components described herein. Each system component can also be used in combination with other system components.

While the invention has been described in terms of various specific embodiments, those skilled in the art will recognize that the invention can be practiced with modification within the spirit and scope of the claims.

1. A method of ultrasound imaging comprising:
coupling a first ultrasound probe to a portable ultrasound-imaging device, the probe configured to provide a first predetermined set of functions;
scanning a volume of interest using the portable ultrasound-imaging device at a first transmit power level capability to acquire ultrasound image data;
coupling the portable ultrasound-imaging device to a resource extension device;
processing at least a portion of the ultrasound image data using an ultrasound-imaging device processor; and
processing at least a portion of the ultrasound image data using a resource extension device processor.

2. A method of ultrasound imaging in accordance with claim 1 further comprising communicatively coupling a second ultrasound probe to a portable ultrasound-imaging device, the second probe configured to provide a second predetermined set of functions, the second set of functions including at least one function different than the functions of the first set of functions,

3. A method of ultrasound imaging in accordance with claim 1 wherein scanning a volume of interest comprises storing the acquired ultrasound image data in a memory of the ultrasound-imaging device in a first data format.

4. A method of ultrasound imaging in accordance with claim 3 wherein processing at least a portion of the ultrasound image data using a resource extension device processor comprises storing the acquired ultrasound image data in a memory of the extension device processor in a second data format.

5. A method of ultrasound imaging in accordance with claim 1 wherein scanning a volume of interest comprises scanning a volume of interest using at least one of a B-mode imaging scan, a continuous wave scan, and a pulsed Doppler imaging scan.

6. A method of ultrasound imaging in accordance with claim 1 wherein coupling the portable ultrasound-imaging device to a resource extension device comprises coupling at least one of an ultrasound-imaging device data path to a resource extension device data path and an ultrasound-imaging device power path to a resource extension device power path.

7. A method of ultrasound imaging in accordance with claim 1 wherein coupling the portable ultrasound-imaging device to a resource extension device comprises automatically synchronizing the images stored in the portable ultrasound-imaging device with images stored in at least one of the resource extension device and a picture archiving communication system (PACS).

8. A method of ultrasound imaging in accordance with claim 1 wherein coupling the portable ultrasound-imaging device to a resource extension device comprises automatically downloading the images stored in the portable ultrasound-imaging device and uploading a list of patients to be scanned including demographic data relating to each patient and a scan protocol.

9. A method of ultrasound imaging in accordance with claim 1 further comprising scanning the volume of interest at a second transmit power level capability when the ultrasound-imaging device is coupled to the resource extension device, the second transmit power level capability being greater than the first transmit power level capability.

10. A method of ultrasound imaging in accordance with claim 1 further comprising scanning the volume of interest at a second image processing level capability when the ultrasound-imaging device is coupled to the resource extension device, the second image processing level capability being greater than the first image processing level capability.

11. An ultrasound-imaging system configured to operate in a first resource extended mode and a second portable mode, comprising:
an ultrasound-imaging device comprising an input port that is configured to receive at least one of a plurality of probes, each probe having at least one function that is different from a function of each other of said plurality of probes;
a resource extension device removably couplable to said ultrasound-imaging device for at least one of adding an ultrasound-imaging capability to said ultrasound-imaging device and modifying an ultrasound-imaging capability of said ultrasound-imaging device; and
a display for outputting ultrasound images.

12. A portable ultrasound-imaging system in accordance with claim 11 configured to operate in at least one of a first mode and a second mode when said ultrasound-imaging device is coupled to said resource extension device.

13. A portable ultrasound-imaging system in accordance with claim 11 configured to operate in a second mode when said ultrasound-imaging device is decoupled from said resource extension device.

14. A portable ultrasound-imaging system in accordance with claim 11 wherein said ultrasound-imaging device is configured to detect when said ultrasound-imaging device is coupled to said resource extension device.

15. A portable ultrasound-imaging system in accordance with claim 14 wherein upon coupling said ultrasound-imaging device to said resource extension device said ultrasound-imaging device is configured to automatically:
download image data from said ultrasound-imaging device to at least one of said resource extension device and an image archive;
upload data relating to a patient to be scanned including at least one of a patient demographic data and an imaging protocol; and
synchronize at least one of data files, software updates, and firmware updates between said ultrasound-imaging device and said resource extension device.

16. A portable ultrasound-imaging system in accordance with claim 11 wherein said ultrasound-imaging device comprises a transmitter configured to receive transmit power from a power supply on-board said ultrasound-imaging device and to receive transmit power from a power supply located within said resource extension device.

17. A portable ultrasound-imaging system in accordance with claim 16 wherein said power supply located within said resource extension device is capable of providing a greater transmit power than said power supply located on-board said ultrasound-imaging device.

18. A portable ultrasound-imaging system in accordance with claim 11 configured to identify a type of probe coupled to said input port.

19. A portable ultrasound-imaging system in accordance with claim 11 configured to display a type of probe coupled to said input port.

20. A portable ultrasound-imaging system in accordance with claim 11 further comprising a motor controller circuit configured to drive a motor of a 3D mechanical probe.

21. A portable ultrasound-imaging system in accordance with claim 11 wherein said resource extension device comprises a processor configured to process at least one of real-time ultrasound data during a scan, ultrasound data stored on-board said ultrasound-imaging device, and ultrasound data stored on said resource extension device.

22. A portable ultrasound-imaging system in accordance with claim 11 wherein said resource extension device comprises a display for outputting at least one of real-time ultrasound data during a scan, ultrasound data processed by said ultrasound-imaging device, and ultrasound data processed by said resource extension device.

23. A portable ultrasound-imaging system in accordance with claim 11 further comprising a connector configured to couple said ultrasound-imaging device and said resource extension device, such that at least one of an ultrasound-imaging device data path is coupled to a resource extension device data path and an ultrasound-imaging device power path is coupled to a resource extension device power path.

24. A portable ultrasound-imaging system in accordance with claim 23 wherein said mating connector is configured to couple said ultrasound-imaging device and said resource extension device through a data network.

25. A portable ultrasound-imaging system in accordance with claim 11 wherein a resource extension device processor capability is greater than an ultrasound-imaging device processor capability.
